(19) United States
(12) Patent Application Publication
(10) Pub. No.: US 2007/0016148 A1
(43) Pub. Date: Jan. 18, 2007

(54) Winged needle assembly

(76) Inventors: Yoshiharu Iwase, Osaka (JP); Hiroyuki Nakagami, Osaka (JP)

Correspondence Address: KUBOVCIK & KUBOVCIK, SUITE 710, 900 17TH STREET NW, WASHINGTON, DC, 20006, US

(21) Appl. No.: 11/434,748

(22) Filed: May 17, 2006

Related U.S. Application Data
(60) Provisional application No. 60/706,025, filed on Aug. 8, 2005.

(30) Foreign Application Priority Data
May 19, 2005 (JP) 2005-146977

(51) Int. Cl. A61M 25/00

(52) U.S. Cl. 604/264

(57) ABSTRACT

A winged needle assembly includes a cannula; a needle hub; a protector of approximately cylindrical shape externally fitted onto the hub; and a pair of wings fixed to the outer peripheral surface of the protector; the hub being allowed, due to the resilient force of a spring, to slide from a first position where the distal end of the cannula protrudes beyond the distal end side of the protector to a second position where the cannula is accommodated in the protector, wherein: the spring is arranged on the outer periphery of the hub and is fixed in the axial direction between the proximal end of the protector and the proximal end of the hub, and first engaging means for engaging the hub at the first position so that it can be disengaged and second engaging means for engaging the hub at the second position so that it cannot be disengaged are provided.

### TECHNICAL FIELD

This invention relates to a winged needle assembly provided with a mechanism which is capable of accommodating a used winged needle in a protector with a simple operation in order to prevent an accident of erroneously sticking the winged needle after it is used.

### BACKGROUND ART

A conventional medical winged needle has been separated from a protector that protects the winged needle by accommodating it therein. Therefore at the time of accommodating the winged needle in the protector after the use a so-called erroneously sticking accident tends to occur, such as a person engaged in the medical work sticks his finger gripping the protector with the needle tip of the winged needle. The erroneous sticking accident could cause the person engaged in the medical work to be infected with AIDS and hepatitis.

In order to prevent the above erroneously sticking accident, the applicant has proposed an indwelling needle with a protector capable of accommodating the needle cannula after the use by a slide operation, arranging a protector of approximately a cylindrical shape on the outer side of a needle hub that supports the proximal end of the needle cannula (see patent document 1). By using this indwelling needle with wings, the person engaged in the medical work is allowed to accommodate the needle cannula in the protector by sliding the needle hub toward the proximal end side in a state where the wings are fixed to the skin of a patient and, hence, to prevent erroneously sticking accident. With this indwelling needle with wings, however, an operation must be carried out to slide the needle hub toward the proximal end side by the length of the needle cannula that had been protruded at least before the use from the distal end side of the protector at the time of accommodating the needle cannula in the protector.

Therefore, there have been proposed winged needle assemblies which are so as to accommodate the needle cannula with automatically sliding toward the proximal end side by using the repulsive force of the spring arranged in a compressed state (see, for example, patent documents 2 and 3). In these winged needle assemblies, the spring is arranged in the protector, the distal end of the spring is fixed to the distal end of the protector, and the proximal end of the spring is fixed to the needle hub. In the winged needle, the needle hub is locked in a state where the needle cannula protrudes beyond the distal end of the protector. Here, the spring has been held in a state of being compressed in the protector. When the needle hub is unlocked, the spring that had been compressed expands so as to resume its natural length, whereby the needle hub is caused to slide toward the proximal end side of the protector, and the needle cannula is accommodated in the protector.

As the mechanism for locking the needle hub in a state where the needle cannula protrudes beyond the distal end side of the protector, the winged needle assembly uses the mechanism that fixes the needle hub to the protector by protecting a flexible arm provided on the outer side of the needle hub from through a hole perforated in the protector to outward.

The winged needle assembly, further, has a mechanism for locking the flexible arm of the needle hub to the proximal end of the protector in a manner that it cannot to be disengaged (see, for example, patent document 2) and a mechanism for locking the distal end of the needle hub to a flexible engaging finger provided at the proximal end of the protector (see, for example, patent document 3), so that the needle cannula accommodated in the protector is reliably protected in the protector without protruding again from the protector.

In the above winged assembly, however, the diameter of the spring become large consequently so as to arrange the spring in the inside of the protector. Further, the winged needle assembly has the mechanism for locking the needle hub in a state where the needle cannula is protruding beyond the distal end side of the protector and the mechanism for locking the needle hub in a state where the needle cannula is accommodated in the protector, which are both provided at positions to come in contact with the spring arranged in the protector. To avoid a contact with the spring, therefore, the locking mechanism is arranged further on the outer side of the spring causing the diameter of the protector to be further increased.

If the diameter of the protector further increases, healthcare professionals will find it difficult to grip the winged needle assembly and to stick the needle cannula into a patient. As the diameter of the protector increases, further, a step is formed by the difference in the diameter between the needle cannula and the protector. Therefore, the protector becomes an obstacle at the time of placing the winged needle assembly on the patient, and the angle of the needle cannula placed on the patient's skin becomes larger.

[Patent document 1] JP-A-10-85333

[Patent document 2] International Patent Publication No. 2001-522626

[Patent document 3] JP-A-2003-180829

### DISCLOSURE OF THE INVENTION

### Problems to be Solved by the Invention

Therefore, it is an object of this invention is providing a winged needle assembly which is free from the problem of operation at the time of sticking the winged needle into the patient by designing the protector in a small diameter with a mechanism in which the needle hub automatically slides toward the proximal end side so that the needle cannula is accommodated in the protector by using the repulsive force of a spring arranged in a compressed state.

### Means for Solving the Problem

Through keen study, the present inventors have discovered that the above problems can be solved by arranging the mechanism for locking the needle hub in a state where the needle cannula is protruding beyond the distal end side of the protector and the mechanism for locking the needle hub in a state where the needle cannula is accommodated in the protector, at positions where they will not come in contact with the spring as a result of arranging the spring on the proximal end side of the protector instead of traditionally arranging the spring inside the protector, and has arrived at the present invention.

That is, this invention is concerned with:

(1) A winged needle assembly comprising a needle cannula; a needle hub having a distal end for holding the proximal end of said needle cannula, an intermediate portion and a proximal end; a approximately cylindrical shape protector externally fitted onto the distal end and onto the intermediate portion of said needle hub, and a pair of wings fixed to the outer peripheral surface of the distal end of said protector; said needle hub being allowed to slide along the inner peripheral surface of said protector due to the resilient force of a spring from a first position where the distal end of said needle cannula protrudes beyond the distal end side of said protector to a second position where said needle cannula is accommodated in said protector, characterized in that:

the proximal end of said needle hub has an outer diameter larger than an inner of said protector and is arranged on the proximal end side of the assembly than position of said protector;

said spring is arranged on the outer periphery of the intermediate portion of the needle hub and is fixed in the axial direction between the proximal end of said protector and the proximal end of said needle hub along an axial direction of the hub; and

said needle hub and said protector are provided with first engaging means for engaging said needle hub at the first position in a manner that it can be disengaged and second engaging means for engaging said needle hub at the second position in a manner that it cannot to be disengaged;

(2) A winged needle assembly according to claim 1, wherein said first engaging means comprises a first engaging portion provided at the proximal end of said needle hub and a first engaged portion that engages with said first engaging portion provided at the proximal end of said protector, and said second engaging means comprises a second engaging portion provided at the proximal end of said protector and a second engaged portion that engages with said second engaging portion provided at the distal end of said needle hub;

(3) A winged needle assembly according to claim 2, wherein the first engaging portion of said first engaging means is a projection protruding outward which is provided at the distal end of an arm extending toward the distal end side from the outer peripheral surface of the proximal end of said needle hub, and the first engaged portion of said first engaging means is a hole or a recessed portion provided at the proximal end of said protector;

(4) A winged needle assembly according to claim 3, wherein said first engaging portion and the first engaged portion are provided each in a number of two symmetrically with respect to the axis of said needle hub on the same plane as the bevel forming the blade tip of said needle cannula;

(5) A winged needle assembly according to claim 2, wherein the second engaging portion of said second engaging means is a flange protruding inward which is provided on the proximal end of said protector, and the second engaged portion of said second engaging means comprises a protruding portion protruding outward which is provided on the distal end of said needle hub and a groove portion having a length equal to the length in the axial direction of said flange provided on the proximal end side of said protruding portion or a length which is slightly greater than the length thereof in the axial direction; and

(6) A winged needle assembly according to claim 5, wherein the flange which is said second engaging portion is an annular flange provided on the inner peripheral surface of said protector over the whole circumference thereof, and the groove which is said second engaged portion is an annular groove provided in the outer peripheral surface of said needle hub over the whole circumference thereof.

### Effect of the Invention

In the winged needle assembly of the invention, the spring which is arranged on the outer periphery of the proximal end of the needle hub protruded from the proximal end side of the protector make it possible to design the protector in a smaller diameter than that of the conventional winged needle assembly having a spring arranged inside the protector. Therefore, a person engaged in the medical work finds it easy to grip the winged needle assembly and easy to stick the needle cannula into the patient. This further decreases a step caused by a difference in the diameter between the needle cannula and the protector, and the angle of the needle cannula placed on the patient's skin does not become larger relative to the skin at the time when the winged needle assembly is placed on the patient.

Further, arrangement of the spring as described above makes it possible to arrange the mechanism (first engaging means) for locking the needle hub in a state where the needle cannula is protruding beyond the distal end side of the protector and the mechanism (second engaging means) for locking the needle hub in a state where the needle cannula is accommodated in the protector at positions where they will not come in contact with the spring, and these mechanisms need not be provided on the outer side of the spring. Therefore, the diameter of the protector does not become further great.

In the winged needle assembly of the present invention, further, the first engaging means for locking the needle hub in a state where the needle cannula is protruding from the distal end side of the protector, is provided on the proximal end side of the protector. At the time of sticking the winged needle into the patient, therefore, it does not happen that the person engaged in the medical work gripping the wing touches the first engaging means with his fingers which may undesirably release the engagement causing the needle cannula to be accommodated in the protector. Upon providing the first engaging means in a plural number, the effect can be further enhanced for preventing the erroneous operation of the first engaging means. By providing the first engaging means at the above position, further, the protector can be designed in a minimum diameter at the position of near the wings that are gripped by the person engaged in the medical work at the time of sticking the winged needle into the patient, and the operability of the winged needle can be further improved.

### BEST MODE FOR CARRYING OUT THE INVENTION

A preferred embodiment of the invention will now be described with reference to the drawings. However, the invention is in not limited to these.

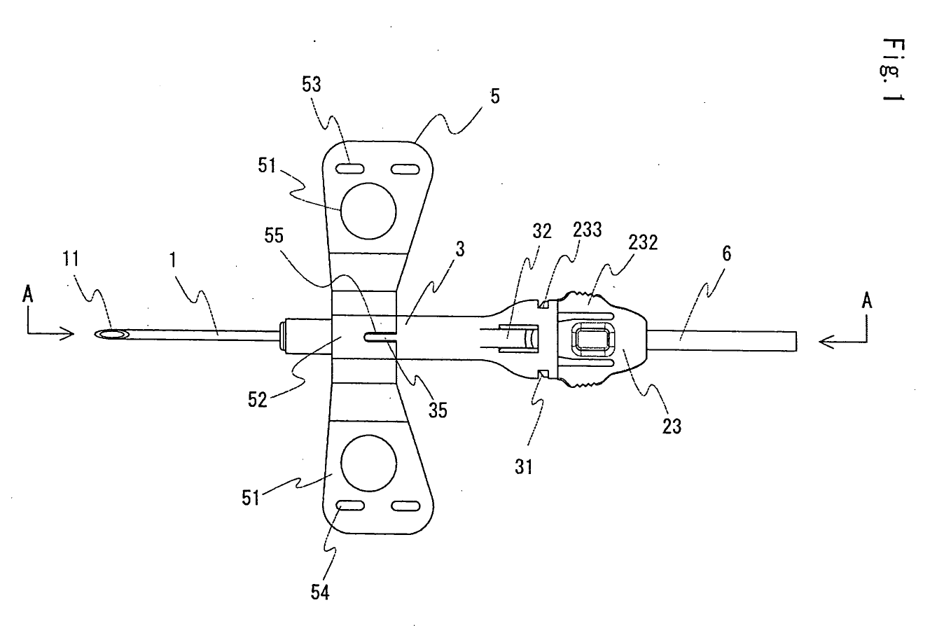

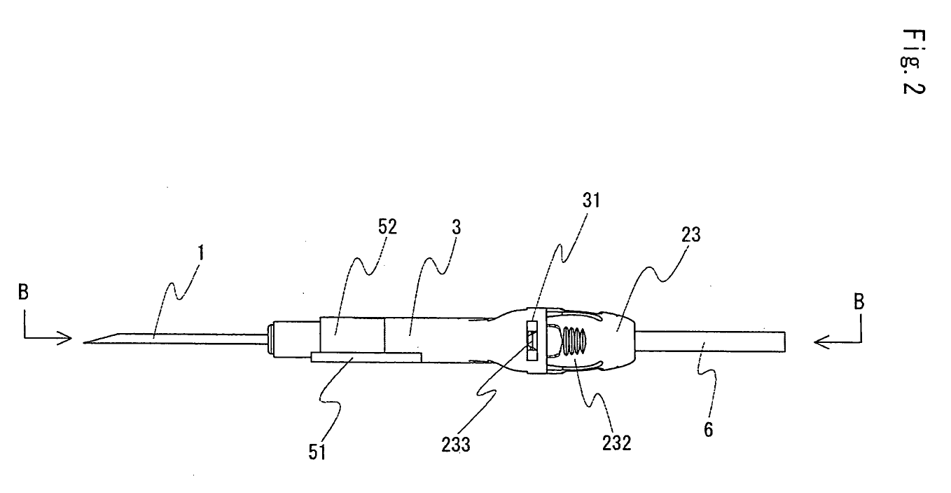

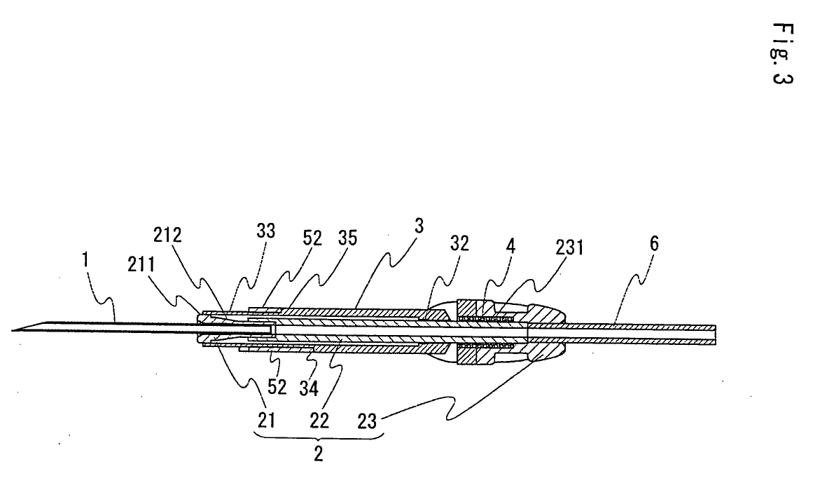

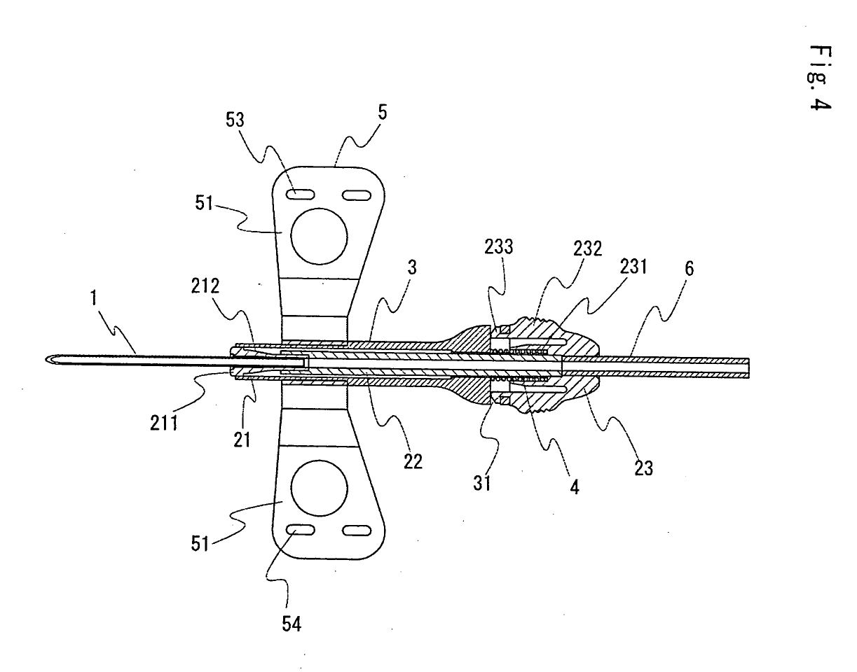

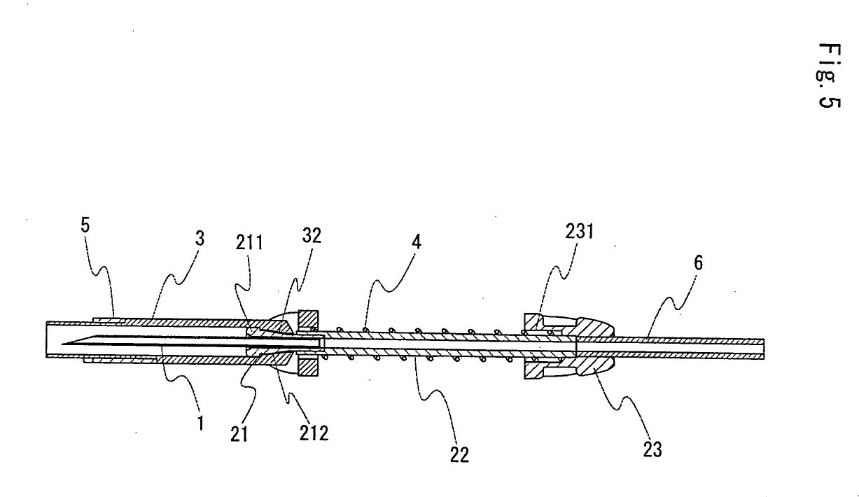

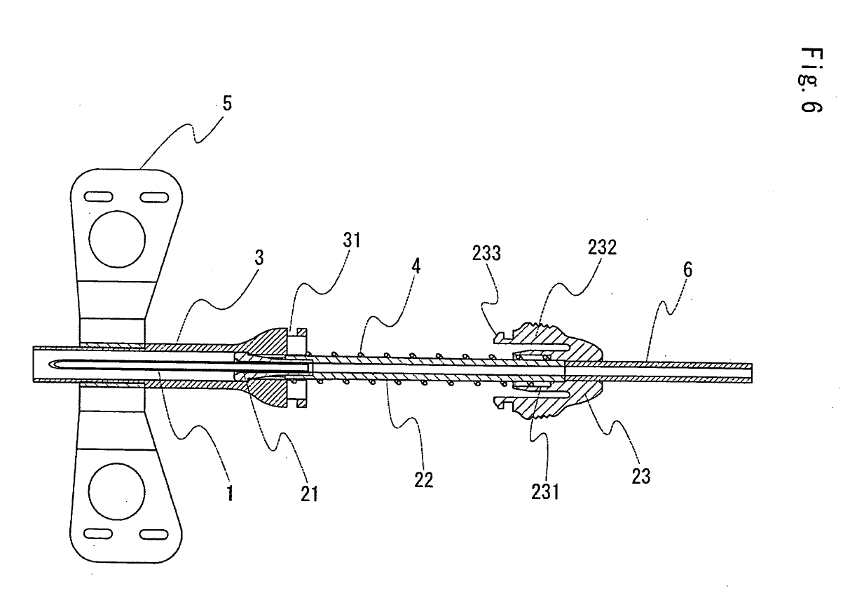

FIG. 1 is a top view illustrating an embodiment of the winged needle assembly of the present invention, and FIG. 2 is a side view of the winged needle assembly shown in FIG. 1. Further, FIG. 3 is a sectional view along the line A-A of the winged needle assembly shown in FIG. 1, and FIG. 4 is a sectional view along the line B-B of the winged needle assembly shown in FIG. 2. FIGS. 1 to 4 are views illustrating a state of prior to using the winged needle assembly of the invention and where the needle cannula is protruding from the distal end side of the protector.

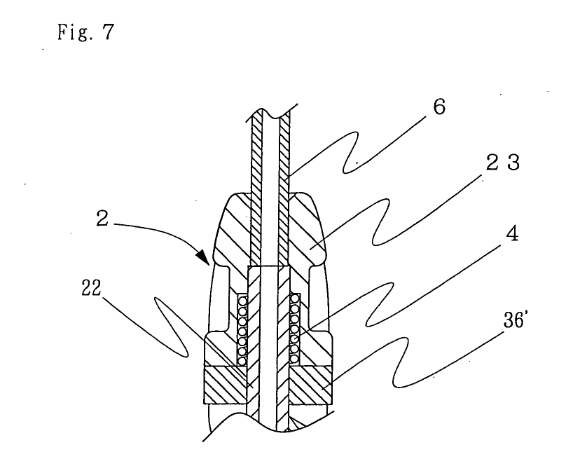

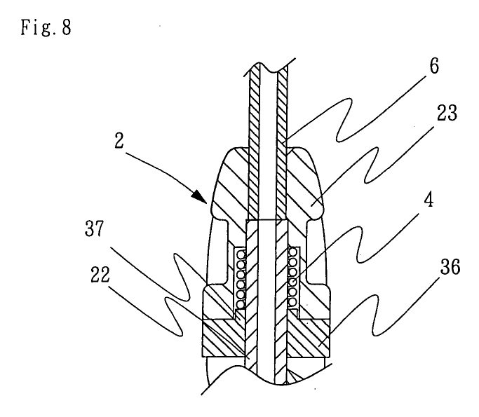

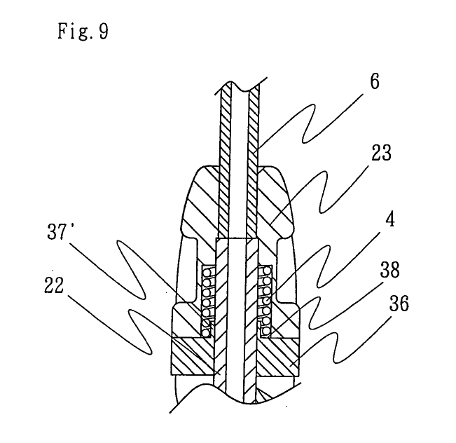

On the other hand, FIGS. 5 and 6 are views illustrating a state of after the use of the winged needle assembly shown in FIGS. 1 to 4 and where the needle cannula is accommodated in the protector. FIG. 5 is a sectional view illustrating the state of after the use of the winged needle assembly shown in FIG. 3, and FIG. 6 is a longitudinal sectional view illustrating the state of after the use of the winged needle assembly shown in FIG. 4.

Referring to FIGS. 1 to 4, the winged needle assembly of the present invention includes a needle cannula 1, a needle hub 2 for holding the proximal end of the needle cannula 1, a protector 3 of approximately a cylindrical shape externally fitted onto the needle hub 2, and a wing 5 fixed to the outer peripheral surface of the distal end of the protector 3.

In the winged needle assembly of the invention, the distal end side stands for the side (left side in the drawing) that is sticked into the patient and the proximal end side means for the side (right side in the drawing) that is connected to the tube 6.

The needle cannula 1 of the present invention is a hollow needle having a sharp blade tip 11 at the distal end. As the material of the needle cannula 1, there can be used a metal such as a stainless steel, aluminum, titanium or an alloy thereof, or a synthetic resin such as polycarbonate, ABS or the like. The needle cannula 1 has a diameter of, usually, 0.4 to 2.1 mm and a thickness of 0.24 to 1.73 mm, however, these sizes can be set to lie outside the above ranges to meet the use. The length of the needle cannula 11 is such that the proximal end is held by the needle hub 2 and a portion protruding from the distal end side of the protector 3 in a state of before the use is long enough for being sticked into the patient's blood vessel. Concretely, the portion protruding from the protector 3 has a length of, preferably, 15 to 32 mm. The overall length of the needle cannula 11 is suitably set by taking into consideration the length of a connection portion of the proximal end of the needle cannula 11 and the needle hub 2, and the position of the needle hub 2 arranged in the protector in a state of before the use, in addition to the length of the portion that protrudes.

The proximal end of the needle cannula 1 is held by the distal end of the needle hub 2. The needle hub 2 is a member of a approximately cylindrical shape formed with a synthetic resin having flexibility, such as polypropylene, polyester or polyethylene. The proximal end of the needle cannula 1 is inserted in a cavity of the needle hub 2 at the distal end, and is fixed thereto so will not to be removed by the adhesion using an adhesive, high-frequency melt-adhesion, or the like. The inner cavity of the needle cannula 1 and the inner cavity of the needle hub 2 in which the needle cannula 1 is not inserted are fluid-communicated with each other.

A tube 6 is connected to the proximal end side of the needle hub 2, said tube 6 is continuous to a medical component that is used for picking up or injecting a fluid. The needle hub 2 and the tube 5 are connected together by a known method such as adhesion by using an adhesive, melt-adhesion, fitting, or the like.

Referring to FIG. 4, the needle hub 2 comprises a distal end 21 for holding the proximal end of the needle cannula 1, a proximal end 23 to which the tube 6 is connected, and an intermediate portion 22 for connecting the distal end 21 and the proximal end 23 together. The needle hub 2 may be the one in which the distal end 21, the intermediate portion 22 and the proximal end 23 are integrally fixed together by such a method as adhesion, or may be the one which as a whole is integrally formed by a conventional method such as injection-molding.

A protector 3 of approximately a cylindrical shape formed by using a synthetic resin such as vinyl chloride or polycarbonate is externally fitted onto the distal end 21 and onto the intermediate portion 22 of the needle hub 2. The protector 3 has a cavity in which the needle hub 2 slides along the inner peripheral surface of the protector 3. The sliding can be achieved by contacting the needle hub 2 with the inner peripheral surface of the protector 3 at least at the distal end thereof and another portion thereof. By taking the operability of the winged needle assembly into consideration, however, it is preferable that the inner diameter of the protector 3 is set to be slightly greater than the outer diameter of the needle hub 2 in order to suppress the outer diameter of the protector 3 as much as possible.

The needle hub 2 can slide in the protector 3 from a first position where the blade tip 11 at the distal end of the needle cannula 1 protrudes beyond the distal end side of the protector 3 up to a second position where the whole needle cannula 1 inclusive of the blade tip 11 is accommodated in the protector 3. The proximal end 23 of the needle hub 2 has an outer diameter greater than the inner diameter of the protector 3. At the first position, the proximal end 23 of the needle hub 2 is disposed on the proximal end side from the protector 3. At the second position as shown in FIG. 5, on the other hand, not only the proximal end 23 of the needle hub 2 but also the intermediate portion 22 as a whole are protruding from the proximal end side of the protector 3. At the second position, further, the distal end 21 of the needle hub 2 and the proximal end of the needle cannula 1 may protrude from the proximal end side of the protector 3. From the sanitary point of view, however, it is preferable that the needle cannula 1 is not directly exposed to the open atmosphere.

The needle hub 2 automatically slides from the first position to the second position by the elastic force of the spring 4 disposed on the outer circumference of the needle hub 2. It is preferable that the spring 4 is a coil spring made of a metal such as a spring steel or a stainless steel. It is further desired that the inner cavity of the coil spring 4 has a diameter which is slightly greater than the outer diameter of the needle hub 2, and that the spring is arranged on the outer circumference of the needle hub 2 being closely contacted thereto to such a degree that will not limit the expansion and contraction of the spring. The coil spring 4 is arranged surrounding the outer circumference of the needle hub 2 and does not deviate in the radial direction.

Further, the spring 4 is fixed in the axial direction between the proximal end of the protector 3 and the proximal end 23 of the needle hub 2. Therefore, the outer diameter of the coil spring 4 is set to be greater than the inner diameter of the protector 3. The proximal end 23 of the needle hub 2 may be provided with a protruding portion 231 that protrudes toward the distal end side so as to hold the proximal end of the spring 4 between said proximal end and the intermediate portion 22.

As shown in FIG. 4, the spring 4 is held between the protector 3 and the proximal end 23 of the needle hub 2 when the needle hub 2 is engaged with the protector 3 by the first engaging means that is be described later. In a state where the spring 4 is stretched as shown in FIG. 6, on the other hand, the needle hub 2 is slid toward the proximal end side up to a position where the blade tip 11 of the needle cannula 1 is completely accommodated in the protector 3.

In either the contracted state or the stretched state, the spring 4 as a whole is positioned on the proximal end side away from the protector 3. With the spring 4 being arranged as described above, it is allowed to design the protector 3 in a diameter very smaller than that of the conventional winged needle assembly in which the spring is arranged in the protector, and the winged needle assembly features improved operability.

The needle hub 2 and the protector 3 are provided with first engaging means for engaging the needle hub 2 at the first position in a manner that it can be disengaged and second engaging means for engaging the needle hub 2 at the second position in a manner that it cannot to be disengaged. In the winged needle assembly of this invention in which the spring 4 is arranged on the proximal end side of the protector 3, there is no probability that the spring 4 comes in contact with the first engaging means and the second engaging means that work to engage the needle hub 2 with the protector 3. The spring 4 needs not be provided on a further outer side so as not to come into contact with the first engaging means and the second engaging means, and the diameter of the protector 3 needs not be increased.

By using the winged needle assembly of the invention in which the needle hub 2 is engaged at the first position by the first engaging means, the needle hub 2 is prevented from being pushed toward the proximal end side at a moment when the needle cannula 1 is sticked into the patient and, besides, the needle hub 2 is prevented from being slid to the proximal end side by the resilient force of the spring 4. After the winged needle assembly has been used, the first engaging means is released, whereby the needle hub 2 is slid toward the proximal end side by the resilient force of the spring and the needle cannula 1 is accommodated in the protector 3.

In the winged needle assembly, further, the needle hub 2 is engaged at the second position by the second engaging means preventing the needle hub 2 from being slid toward the distal end side or the proximal end side. Therefore, the needle cannula 1 is not exposed again from the state where it is contained in the protector 3.

As the first engaging means, the means comprising, for example, a first engaging portion provided at the proximal end 23 of the needle hub 2 and a first engaged portion provided at the proximal end of the protector 3 can be used. Concretely as shown in FIGS. 1 and 4, the proximal end 23 of the needle hub 2 is provided with a flexible arm 232 that extends toward the distal end side from the outer peripheral surface of the proximal end 23 and, and, further, the distal end of the arm 232 is provided with a projection 233 protruding outward as the first engaging portion. Further, the proximal end of the protector 3 is provided with a through hole 31 or a recessed portion which is opening inward as the first engaged portion. The arm 232 is so formed as to be urged outward, and the needle hub 2 is brought into engagement with the protector 3 as the projection 233 protrudes into the hole 31 from the inside of the protector 3.

To release the engagement of the first engaging means after the winged needle assembly has been used, the projection 233 is disengaged from the inside of the hole 31 with pushing down the projection 233 caused by pushing inward of the arm 232. The arm 232 may be provided as only one part at the proximal end 23 of the needle hub 2. As shown in the figs, however, it is preferable that the arms 232 are provided as two parts symmetrically with respect to the axis of the needle hub 2 on a same plane with the bevel that forms the blade tip 11 of the needle cannula 1. When the arms 232 are provided in a number of two, further, the projections 233 and the holes 31 are provided each in a number of two correspondingly. The person engaged in the medical work who use the winged needle assembly grips the two arms 232 by the thumb and index finger of his hand after the use of the winged needle assembly to easily release the first engaging means. Here, since the projection 231 is provided at the proximal end 23 of the needle hub 2, the arm 232 is reliably prevented from coming in contact with the spring 4 when the first engaging means is released and the spring 4 is not prevented from stretching.

In the conventional winged needle assemblies, the first engaging means is, in many cases, provided near the wing of the winged needle, and the locking tends to be undesirably released at the time of operating the winged needle. The portion where the first engaging means is provided causes an increase in the diameter of the protector leaving a problem of poor operability.

By using the winged needle assembly of this invention in which the first engaging means is provided on the proximal end side of the protector, however, the person engaged in the medical work who grips the wing is prevented from undesirably touching the first engaging means by his fingers causing the locking to be released at the time of sticking the winged needle into the patient. Further, provision of the first engaging means at the above position makes it possible to design the protector in a minimum diameter near the wing that is gripped by the person engaged in the medical work at the time of sticking the winged needle into the patient, contributing to further improving the operability of the winged needle.

As the second engaging means, there is used means comprising, for example, a second engaging portion provided at the proximal end of the protector 3 and a second engaged portion provided at the distal end 21 of the needle hub 2. Concretely as shown in FIGS. 3 and 5, the proximal end of the protector 3 is provided with a flexible flange 32 that protrudes inward as the second engaging portion. Further, the distal end 21 of the needle hub 2 is provided with a protruding portion 211 protruding outward and with a groove portion 212 arranged on the proximal end side of the protruding portion 211 as the second engaged portion. The groove portion 212 is designed to have a length equal to the length in the axial direction of the flange 32 or is slightly greater than the length thereof in the axial direction. The flange 32 is formed so as to be biased inward. By fitting into the groove portion 212, the distal end side of the flange comes in contact with the protruding portion 211 and the proximal end side of the flange comes in contact with the edge on the proximal end side of the groove portion 212 and, thus, the needle hub 2 engages with the protector 3. The groove portion 212 may be provided in the distal end 21 of the needle hub 2, or may be formed by fixing together the distal end 21 and the intermediate portion 22 of the needle hub 2 as shown in FIGS. 3 and 5.

The flange 32 may be provided at one place or at a plurality of places along the inner peripheral surface of the protector 3, or may be an annular flange provided of the inner peripheral surface thereof over the whole circumference. The groove portion 212, is also provided so as to be corresponded to the shape of the flange 32, and may be provided at one place or at a plurality of places along the outer peripheral surface of the needle hub 2, or may be an annular groove provided in the outer peripheral surface thereof over the whole circumference.

A pair of wings 5 are secured onto the outer peripheral surface of the protector 3. The wings 5 are secured by a known method such as adhesion or fitting. The wings 5 are formed by using a flexible material such as polyolefin, polyvinyl chloride, or the like. The wings 5 include two pieces of flexible wing 51 and a cylindrical base 52 externally fitted onto the protector. One vane may be provided with a protuberance 53 on the upper surface thereof and the other vane 51 may be provided with a recessed portion 54 in the upper surface thereof so as to be fitted to the protuberance 53 each in a number of one or in a plural number, so that the upper surfaces thereof can be joined together and fixed at the time of sticking the winged needle into the patient.

It is desired that the wings 5 are externally fitted onto the protector 3 in a manner that the wing 51 are arranged on the same plane as the blade tip 11 of the needle cannula 1, and are secured thereto so as not to rotate from the positions thereof. As shown, for example, in FIGS. 1 and 3, a contracted-diameter portion 33 is formed on the outer peripheral surface of the protector 3 on the distal end side thereby to form a step portion 34, and a protruded portion 35 is provided on the outer peripheral surface of the contracted-diameter portion 33 extending from the stepped portion 34 toward the distal end side. Further, the wings 5 are provided with a groove portion 55 that fits to the protruded portion 35 of the base 52. The protruded portion 35 is fitted into the groove portion 55 at the time of externally fitting the base 52 onto the contracted-diameter portion 33 of the protector 3 to prevent the rotation of the wings 5 relative to the protector 3. According to another embodiment, though not illustrated, the protector 3 and the base 52 of wings 5 are formed in a non-circular shape in cross section to easily prevent the rotation of the wings 5.

To produce the winged needle assembly of the invention, first, the proximal end of the distal end 21 and the intermediate portion 22 of the needle hub 2 are fixed together by adhesion or fitting. Next, the needle hub 2 is inserted in order of the intermediate portion 22 and the distal end 21 from the distal end side of the protector 3 onto which the wings 5 have been externally fitted. The end of the intermediate portion 22 on the proximal end side is caused to protrude from the proximal end side of the protector 3, and the spring 4 is externally fitted onto the intermediate portion 22 in a state where the needle hub 2 is arranged on the proximal end side away from the first position. Thereafter, the proximal end 23 of the needle hub 2 is fixed to the proximal end side of the intermediate portion 22 by adhesion or fitting, and the spring 4 is fixed in the axial direction between the proximal end of the protector 2 and the proximal end 23 of the needle hub 2. When the proximal end 23 of the needle hub 2 is provided with the protruding portion 231, the proximal end of the spring 4 is held between the intermediate portion 22 of the needle hub 2 and the protruding portion 231.

After the distal end 21, intermediate portion 22 and proximal end 23 of the needle hub 2 are reliably fixed in the above state, the needle hub 2 is pushed toward the distal end side relative to the protector 3, and the needle hub 2 and the protector 3 are engaged together by the first engaging means. Thereafter, the proximal end of the needle cannula 11 is inserted from the side of the distal end 21 of the needle hub 2 and is fixed thereto, while the end of the tube 6 is inserted from the side of the proximal end 23 of the needle hub 2 and is fixed thereto, thereby to complete the winged needle assembly of the invention. The winged needle assembly may, further, have a cap (not shown) fitted onto the needle cannula 1 to prevent the occurrence of undesired sticking accident by the blade tip 11 of the needle cannula 1 before the use.

Next, described below with reference to the drawings is how to use the winged needle assembly of the present invention.

First, the cap is removed from the needle cannula 1 of the winged needle assembly, and the blade tip 11 of the needle cannula 1 is sticked into the patient in a state where the needle hub 2 is engaged by the first engaging means at the first position at which the distal end of the needle cannula 1 protrudes from the distal end side of the protector 4. After the medical treatment has been finished, the arms 232 provided at the proximal end 23 of needle hub 2 of the winged needle assembly are pushed from the outer sides to release the first engaging means, whereby the needle hub 2 slides toward the proximal end side due to the spring 4, and the needle cannula 1 is accommodated in the protector 3. When the needle hub 2 slides up to the second position where the needle cannula 1 is completely accommodated in the protector, the needle hub 2 is engaged by the second engaging means in a manner that it is not disengaged, and the needle cannula 1 is prevented from being exposed again from the protector 3.

### BRIEF DESCRIPTION OF THE DRAWINGS

[FIG. 1] is a top view illustrating an embodiment of the winged needle assembly of the present invention.

[FIG. 2] is a side view of the winged needle assembly shown in FIG. 1.

[FIG. 3] is a sectional view along the line A-A of the winged needle assembly shown in FIG. 1.

[FIG. 4] is a sectional view along the line B-B of the winged needle assembly shown in FIG. 2.

[FIG. 5] is a sectional view illustrating the state of after the use of the winged needle assembly shown in FIG. 3.

[FIG. 6] is a longitudinal sectional view illustrating the state of after the use of the winged needle assembly shown in FIG. 4.

### DESCRIPTION OF REFERENCE NUMERALS

1 needle cannula
11 blade tip
2 needle hub
21 distal end
211 protruding portion
212 groove portion
22 intermediate portion
23 proximal end
231 protruding portion
232 arms
233 projection
3 protector
31 hole
32 flange
33 contracted-diameter portion
34 step
35 protruded portion
4 spring
5 wings
51 vanes
52 base
53 protuberances
54 recessed portions
6 tube

1. winged needle assembly comprising a needle cannula; a needle hub having a distal end for holding the proximal end of said needle cannula, an intermediate portion and a proximal end; a approximately cylindrical shape protector externally fitted onto the distal end and onto the intermediate portion of said needle hub, and a pair of wings fixed to the outer peripheral surface of the distal end of said protector; said needle hub being allowed to slide along the inner peripheral surface of said protector due to the resilient force of a spring from a first position where the distal end of said needle cannula protrudes beyond the distal end side of said protector to a second position where said needle cannula is accommodated in said protector, characterized in that:
the proximal end of said needle hub has an outer diameter larger than an inner of said protector and is arranged on the proximal end side of the assembly than position of said protector;
said spring is arranged on the outer periphery of the intermediate portion of the needle hub and is fixed in the axial direction between the proximal end of said protector and the proximal end of said needle hub along an axial direction of the hub; and
said needle hub and said protector are provided with first engaging means for engaging said needle hub at the first position in a manner that it can be disengaged and second engaging means for engaging said needle hub at the second position in a manner that it cannot to be disengaged.

2. A winged needle assembly according to claim 1, wherein said first engaging means comprises a first engaging portion provided at the proximal end of said needle hub and a first engaged portion that engages with said first engaging portion provided at the proximal end of said protector, and said second engaging means comprises a second engaging portion provided at the proximal end of said protector and a second engaged portion that engages with said second engaging portion provided at the distal end of said needle hub.

3. A winged needle assembly according to claim 2, wherein the first engaging portion of said first engaging means is a projection protruding outward which is provided at the distal end of an arm extending toward the distal end side from the outer peripheral surface of the proximal end of said needle hub, and the first engaged portion of said first engaging means is a hole or a recessed portion provided at the proximal end of said protector.

4. A winged needle assembly according to claim 3, wherein said first engaging portion and the first engaged portion are provided each in a number of two symmetrically with respect to the axis of said needle hub on the same plane as the bevel forming the blade tip of said needle cannula.

5. A winged needle assembly according to claim 2, wherein the second engaging portion of said second engaging means is a flange protruding inward which is provided on the proximal end of said protector, and the second engaged portion of said second engaging means comprises a protruding portion protruding outward which is provided on the distal end of said needle hub and a groove portion having a length equal to the length in the axial direction of said flange provided on the proximal end side of said protruding portion or a length which is slightly greater than the length thereof in the axial direction.

6. A winged needle assembly according to claim 5, wherein the flange which is said second engaging portion is an annular flange provided on the inner peripheral surface of said protector over the whole circumference thereof, and the groove which is said second engaged portion is an annular groove provided in the outer peripheral surface of said needle hub over the whole circumference thereof.

7. A winged needle assembly according to claim 1, wherein a cylindrical convex part is formed in the proximal end of the protector; the assembly have the structure which has a cylindrical convex part contacting the spring on the top, or the structure which has a cylindrical convex part 37′ hold in the spring.
